(19) United States
(12) Patent Application Publication
(10) Pub. No.: US 2022/0322965 A1
(43) Pub. Date: Oct. 13, 2022

(54) TRANSFORMING CHEAP SPIROMETERS TO ESTIMATE FLOW-VOLUME GRAPH BY DEEP LEARNING

(71) Applicant: UNIVERSITY OF SOUTH CAROLINA, COLUMBIA, SC (US)

(72) Inventors: SANJIB SUR, CAYCE, SC (US); AAKRITI ADHIKARI, COLUMBIA, SC (US)

(21) Appl. No.: 17/715,561

(22) Filed: Apr. 7, 2022

Related U.S. Application Data
(60) Provisional application No. 63/171,797, filed on Apr. 7, 2021.
(60) Provisional application No. 63/182,257, filed on Apr. 30, 2021.

(51) Int. Cl. A61B 5/087; A61B 5/00; G16H 50/70

(52) U.S. Cl. CPC A61B 5/087; A61B 5/7267; A61B 5/742; A61B 5/0022; G16H 50/70

(57) ABSTRACT

Integrated methodologies and associated devices are provided for performing at-home spirometry tests using improved analysis based on deep learning technology. A deep residual decoder network is established for patient lung function monitoring. Curve learning is conducted with such decoder network based on a patient database of lung function measures. An existing spirometry device may be used for outputting at least one key spirometry indicator and obtaining airflow data from a patient with incident airflow of a sample exhalation resulting in at least one key spirometry indicator from the spirometry device for the patient user. The decoder network processes such spirometry indicator for the patient user to produce flow-volume graphing to extend the capability of the spirometry device for finer-grained, long-term lung function monitoring.

### PRIORITY CLAIM

The present application claims the benefit of priority of U.S. Provisional Patent Application No. 63/171,797, titled Transforming Cheap Spirometers to Estimate Flow-Volume Graph by Deep Learning, filed Apr. 7, 2021, and of U.S. Provisional Patent Application No. 63/182,257, titled Transforming Cheap Spirometers to Estimate Flow-Volume Graph by Deep Learning, filed Apr. 30, 2021, both of which are fully incorporated herein by reference for all purposes.

### BACKGROUND OF THE PRESENTLY DISCLOSED SUBJECT MATTER

Presently disclosed subject matter relates generally to the importance of at-home spirometry, and more particularly, to facilitating at-home spirometry with inexpensive devices.

Flow-volume graphs are important in spirometry tests. Respiratory diseases, such as asthma and COPD, have been a significant public health challenge over decades, and the recent COVID-19 pandemic has worsened the situation. Portable, at-home-use spirometers are effective in continuous monitoring of respiratory syndromes out-of-clinic. Many at-home-use lung function monitoring systems currently exist, but they are either expensive or provide limited spirometry information^{[9;10]}.

For example, a cheap, handheld spirometer like MIR Smart One™ can measure only two out of the seven key spirometry indicators.^{[1]} FIG. 1A represents a handheld MIR Smart One™ device. FIG. 1B represents an MIR Smart One™ Smartphone App. Such device and its App provide only two spirometry parameters: PEFR and FEV1.^{[1]} However, a pulmonologist leverages all key indicators to identify various lung conditions as normal, obstructive, or restrictive, and other respiratory syndromes.

The seven key respiratory indicators are:
1. Peak Expiratory Flow Rate (PEFR): The maximum flow rate during exhalation; healthy adults have a PEFR between 8-10 Liters/second (L/s), and asthma patients, for example, have an average PEFR of 5 L/s or lower^{[2; 3]}.
2. Forced Vital Capacity (FVC): Total air volume exhaled; healthy adults have a capacity between 3.75-5.25 Liters (L), and COPD patients have an average of 2.6 L or lower^{[4]}.
3. Forced Expiratory Volume—1 second (FEV1): Air volume exhaled in the first second of the test; healthy adults exhale 75-80% of their capacity within 1 second, but obstructions in the airways would substantially reduce it^{[4]}.
4-6. Forced Expiratory Flows (FEF25, FEF50, FEF75): The measure of flow rates at 25%, 50%, and 75% of total capacity; used in airway narrowing assessments^{[5]}.
7. Maximal Mid-Expiratory Flow (MMEF): Maximum mid-airflow rate during exhalation; used to diagnose minute airway dysfunctions^{[6]}.

In addition to the spirometry indicators, clinical and high-end spirometers can also provide flow-volume graphs. FIG. 2A represents known, current spirometry tests with an in-clinic version illustrated in the top portion of the Figure, and portable technology illustrated in the bottom illustration of FIG. 2A. FIG. 2B represents exemplary spirometry indicators and flow-volume graphing.

Generally speaking, flow-volume graphing can help visually diagnose a patient's condition. FIG. 3 illustrates some examples of flow-volume graphs for patients with different lung conditions.

Furthermore, flow-volume graph data also indicates if the patient has performed the spirometry test correctly^{[7]}. Thus, pulmonologists use such a graph to not only observe the patient's test performance but also to diagnose the patient's lung condition.

### Summary of the Presently Disclosed Subject Matter

Aspects and advantages of the presently disclosed subject matter will be set forth in part in the following description, may be apparent from the description, or may be learned through practice of the presently disclosed subject matter.

Broadly speaking, the presently disclosed subject matter relates to an approach for performing at-home spirometry tests.

Generally speaking, the presently disclosed subject matter relates to transforming cheap spirometers and emulating the clinical/high-end spirometers otherwise existing for home use.

Presently disclosed subject matter/technology is referenced herein as “SpiroDecoder,” which aims to produce an accurate flow-volume graph from spirometry indicators.

SpiroDecoder is the first-of-a-kind system that can transform the capability of cheap, off-the-shelf spirometer technology that provides limited spirometry indicators by leveraging the recent advancements in deep learning. SpiroDecoder can provide a solution to extend the capability of existing at-home spirometers for finer-grained, long-term lung function monitoring in the post-COVID era.

As would be understood to one of ordinary skill in the art, a straightforward analysis approach could be to use regression or polynomial curve fitting over the seven indicators. However, such approach does not work since the volumes (x-axis) corresponding to the predicted flow rates (y-axis) are unknown.

Instead, SpiroDecoder implements a design of a deep residual decoder network for curve learning based on an open-source CDC database on lung function measures^{[8]}. Then, at run-time, the network takes key indicators as the input and generates a complete flow-volume graph as the output.

One presently disclosed exemplary methodology preferably relates to an integrated methodology for performing at-home spirometry tests using improved analysis based on deep learning technology.

Such exemplary embodiment of integrated methodology preferably comprises establishing a deep residual decoder network for patient lung function monitoring, and conducting curve learning with such decoder network based on a patient database of lung function measures; providing a spirometry device outputting at least one key spirometry indicator and obtaining airflow data from a patient with incident airflow of a sample exhalation resulting in at least one key spirometry indicator from the spirometry device for the patient user; and processing such spirometry indicator for the patient user using the decoder network to produce flow-volume graphing from such spirometry indicator to extend the capability of the spirometry device for finer-grained, long-term lung function monitoring.

It is to be understood from the complete disclosure herewith that the presently disclosed subject matter equally relates to both methodology and corresponding and related apparatus.

One presently disclosed exemplary embodiment relates to a smart device programmed with deep learning technology for operating in accordance with the above-described exemplary integrated methodology in conjunction with a spirometry device to produce flow-volume graphing from spirometry indicators from such spirometry device.

Another presently disclosed exemplary embodiment relates to a device (such as a smart device, smartphone, or device accessing a cloud server) for accessing deep learning technology and for operating in accordance with the above-described exemplary integrated methodology in conjunction with a spirometry device to produce flow-volume graphing from spirometry indicators from such spirometry device.

Another presently disclosed exemplary embodiment relates to methodology for implementing a deep stacked residual learning model to transform the capability of at-home spirometry testing devices using improved analysis based on deep learning technology to produce accurate flow-volume graphing from spirometry indicators, such methodology preferably comprising establishing a decoder network for patient lung function monitoring and having deep stacked residual learning model architecture; conducting curve learning with such decoder network based on spirometry indicators from a relatively large database of diverse patient lung function measures; providing a spirometry device outputting a plurality of key spirometry indicators and obtaining airflow-based data from a patient with incident airflow of a sample exhalation resulting in at least one key spirometry indicator from the spirometry device for the patient user; and processing such spirometry indicator data for the patient using the decoder network to produce flow-volume graphing output from such spirometry indicator input to extend the capability of the spirometry device for finer-grained, long-term lung function monitoring.

Another presently disclosed exemplary embodiment relates to a smart device programmed with a deep stacked residual learning model operating in accordance with the foregoing exemplary methodology to transform the capability of at-home spirometry testing devices by using improved analysis to produce accurate flow-volume graphing based on spirometry indicators from an associated spirometry testing device.

Another presently disclosed exemplary embodiment relates to a device (such as a smart device, a smartphone, or a device which accesses a cloud server) having access to a deep stacked residual learning model operating in accordance with the foregoing exemplary methodology to transform the capability of at-home spirometry testing devices by using improved analysis to produce accurate flow-volume graphing based on spirometry indicators from an associated spirometry testing device.

Other example aspects of the present disclosure are directed to systems, apparatus, tangible, non-transitory computer-readable media, user interfaces, memory devices, and electronic smart devices or the like. To implement methodology and technology herewith, one or more processors may be provided, programmed to perform the steps and functions as called for by the presently disclosed subject matter, as will be understood by those of ordinary skill in the art.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the Figures or stated in the detailed description of said Figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

### BRIEF DESCRIPTION OF THE FIGURES

A full and enabling disclosure of the presently disclosed subject matter, including the best mode thereof, directed to one of ordinary skill in the art, is set forth in the specification, which makes reference to the appended Figures, in which:

FIG. 1A represents a handheld MIR Smart One™ spirometer (prior art), and FIG. 1B represents an MIR Smart One™ Smartphone App (prior art) for use with such spirometer device;

FIG. 2A represents known (prior art) current spirometry tests with an in-clinic version illustrated in the top portion of the Figure, and portable technology illustrated in the bottom illustration of FIG. 2A;

FIG. 2B represents exemplary spirometry indicators and exemplary flow-volume graphing;

FIG. 3 illustrates some examples of flow-volume graphs for patients under different exemplary lung conditions;

FIG. 4A represents exemplary deep residual decoder architecture of the presently disclosed technology;

FIG. 4B shows exemplary network parameters such as for use in the exemplary decoder architecture of present FIG. 4A;

FIG. 5A illustrates two examples of ground-truth flow-volume graphs and predictions from SpiroDecoder compared with a simple regression fit graph; and

FIG. 5B illustrates a similarity score of flow-volume graph predictions (using SpiroDecoder) across a plurality of test samples.

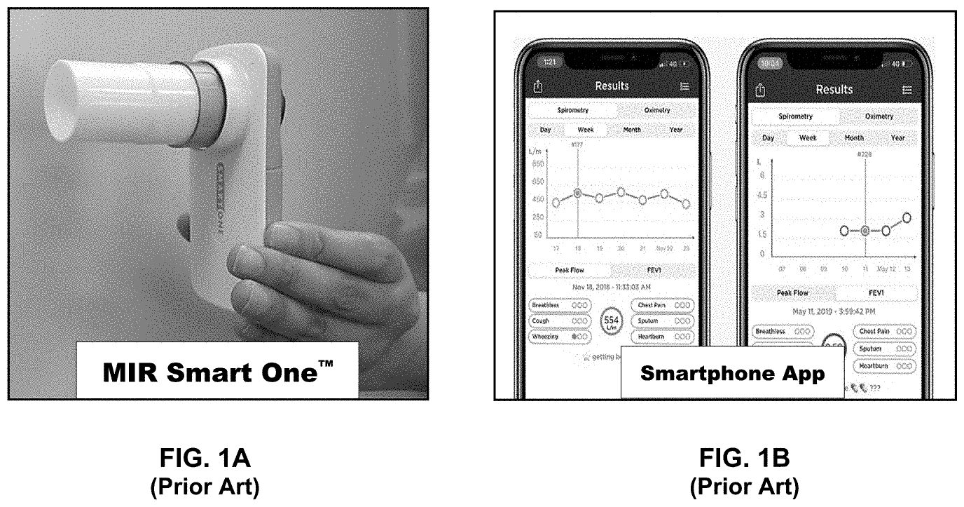

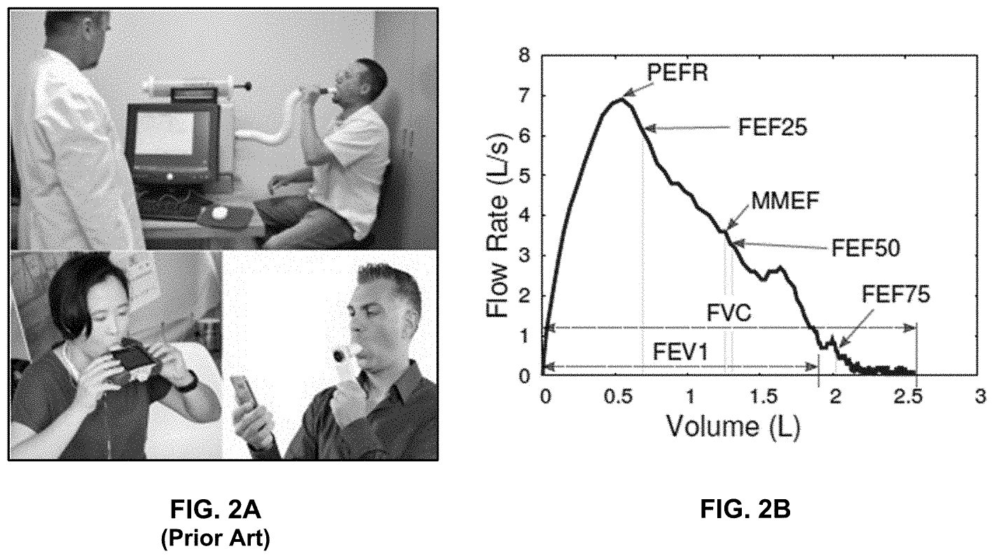

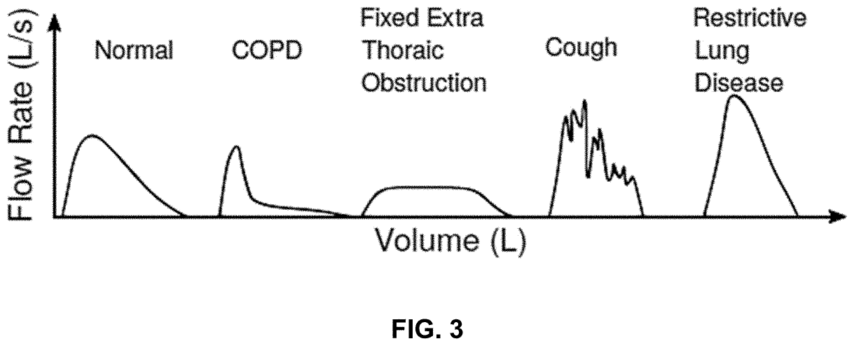

Repeat use of reference characters in the present specification and figures is intended to represent the same or analogous features or elements or steps of the presently disclosed subject matter.

### DETAILED DESCRIPTION OF THE PRESENTLY DISCLOSED SUBJECT MATTER

It is to be understood by one of ordinary skill in the art that the present disclosure is a description of exemplary embodiments only and is not intended as limiting the broader aspects of the disclosed subject matter. Each example is provided by way of explanation of the presently disclosed subject matter, not limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as part of one embodiment can be used with another embodiment to yield a still further embodiment. Thus, it is intended that the presently disclosed subject matter covers such modifications and variations as come within the scope of the appended claims and their equivalents.

The present disclosure is generally directed to the use of technology as an approach to performing at-home spirometry tests.

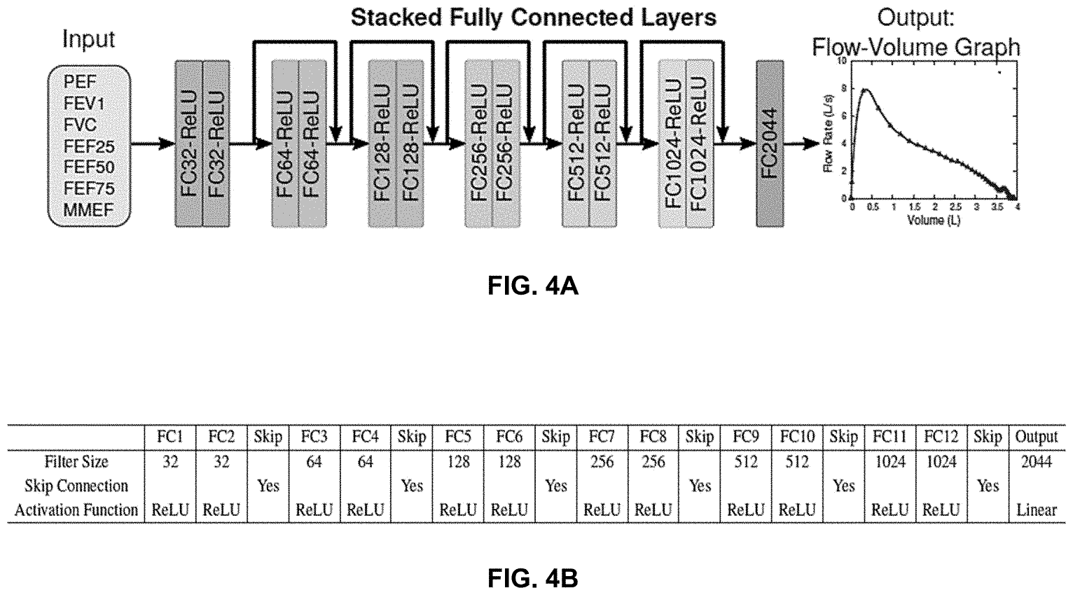

FIG. 4A represents deep residual decoder architecture of SpiroDecoder. Each stage of the decoder employs a fully-connected dense layer that expands the input towards the full flow-volume graph. Since the input size is very small (only 7, for the seven key spirometry indicators) compared to the output size (e.g., 1000, for a flow-volume graph with resolution 0.01 L and maximum 10 L), SpiroDecoder's decoder relies on 13 fully-connected layers stacked on each other. Each layer takes input from its previous layer to expand the compressed input towards the abstract features of the full flow-volume graph.

Such a relatively large number of stacked layers is necessary to expand the decoder's input to output, but it poses a challenge for the backpropagation signal to penetrate the stacks, reach the previous layers, and adapt weights efficiently during training. So, the network could learn suboptimal mapping and produce incorrect flow-volume graphs. To overcome this challenge, SpiroDecoder employs skip connections between the stacked layers that enable not only easier backpropagation but also easier learning of the residual mapping between input and output, ensuring accurate flow-volume graph prediction.

The decoder uses ReLU (Rectified Linear Unit) activation function for its deep neural networks, with ReLU activation used for each stacked layer since the spirometry indicators do not have any negative values. FIG. 4B shows the network parameters of SpiroDecoder (FC=Fully Connected network).

SpiroDecoder's decoder relies on a network loss function to appropriately tune the fully connected layers' weights. To ensure the outliers in the ground-truth data in the CDC database^{[8]} do not affect the network performance significantly, SpiroDecoder uses the Mean Absolute Error (MAE) as the decoder's loss function. MAE calculates the average of the absolute difference between the estimated values and the real values:

$$MAE = \frac{1}{N} {{\sum }^{N}}_{1} \vert {Y}_{i} - {\tilde{Y}}_{i} \vert \qquad ( 1 )$$

where, N is the total number of points, Yᵢ is the observed values, and \tilde{Y}ᵢ is the predicted value.

In one exemplary embodiment, SpiroDecoder's decoder may be implemented with RMSprop optimizer, learning rate of 10⁻⁴, mini-batch size of 32, and ReLU activations.

For the ground-truth data in one embodiment for training the network, one may rely on a large, open-source spirometry database released by the CDC^{[8]}. Such open-source database consists of real but anonymized patients' data collected over one year. As such, it contains a rich deposit of spirometry flow-volume and indicators; in this instance, measured from tests on diseased and healthy individuals from different demographics aged 6 to 79 years. In one embodiment, SpiroDecoder's decoder was trained on 155,000 CDC data. During the run-time of such process, only indicator values were fed in to estimate the complete flow-volume graph.

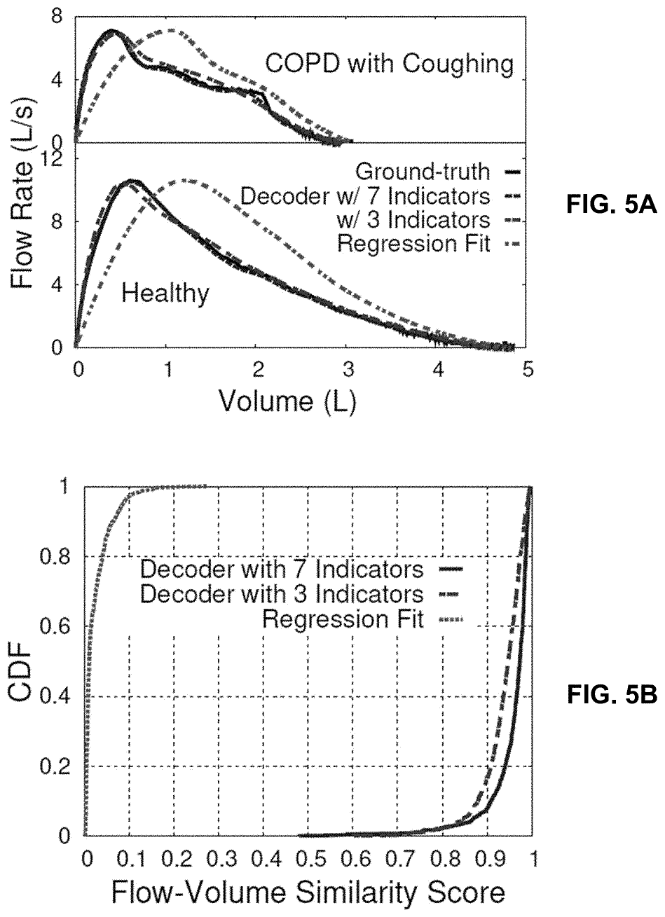

Accuracy of flow-volume graph prediction is represented as follows. FIG. 5A shows two examples of ground-truth flow-volume graphs and predictions from SpiroDecoder and a simple regression fit. To understand the benefit of SpiroDecoder, such regression fit is implemented as a baseline method of parametric regression-based flow-volume graph prediction. Clearly, regression fit fails to identify the correct flow-volume in both the cases. But SpiroDecoder predicts accurate flow-volume for the healthy and diseased cases and works with less than seven indicators too, so that it can be readily used with cheap off-the-shelf spirometers with limited indicators.

FIG. 5B illustrates a similarity score of flow-volume graph predictions (using presently disclosed technology) across a plurality of test samples (in particular, across 871 test samples).

Specifically, to test the performance of SpiroDecoder under a large number of cases, one can find the similarity between predicted and ground-truth flow-volume graphs by measuring normalized cross-correlation on the scale of 0 to 1, where 1 equals the perfect match of the predicted graph with the ground-truth. FIG. 5B shows that the regression fit achieves a similarity score of only 0.06, even at the 90th percentile, indicating that the regression fit method produces incorrect flow-volume graphs. In contrast, SpiroDecoder can predict accurate flow-volume graphs with a median similarity score of 0.97. Even with limited indicators, the median similarity score is still 0.96, indicating the effectiveness of implementing a deep stacked residual learning model to transform the capability of cheap, off-the-shelf spirometers.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims.

### REFERENCES

[1] Medical International Research, “Smart One App,” 2021. [Online]. Available: https://www.spirometry.com/software/mir-smart-one-app/[2]
[2] Ian Gregg, et al., “Peak Expiratory Flow in Normal Subjects,” British Medical Journal, vol. 3, no. 5874, 1973.
[3] William M Tierney, et al., “Assessing Symptoms and Peak Expiratory Flow Rate as Predictors of Asthma Exacerbations” Journal of General Internal Medicine, vol. 19, no. 3, 2004.
[4] Nuvoair, “Do You Know How to Interpret the Results of Your Spirometry Test?” 2021. [Online]. Available: https://bit.ly/2ZHHIZz
[5] Raji Hanieh, et al., “Forced Expiratory Flow at 25-75% as a Marker for Airway Hyper Responsiveness in Adult Patients with Asthma-like Symptoms,” Tanaffos, vol. 17, no. 2, 2018.
[6] Majid Mirsadraee, et al., “Diagnosis of Chronic Obstructive Pulmonary Disease Earlier than Current Global Initiative for Obstructive Lung Disease Guidelines Using a Feasible Spirometry Parameter,” Chronic Respiratory Disease, vol. 10, no. 4, 2013.
[7] Mayank Goel, et al., “SpiroCall: Measuring Lung Function over a Phone Call,” in ACM CHI, 2016.
[8] Center for Disease Control and Prevention, “NHANES Comprehensive Data List,” 2014. [Online]. Available: https://wwwn.cdc.gov/nchs/nhanes/search/datapage.aspx
[9] Microlife, “Asthma Monitor,” 2020. [Online]. Available: https://bit.ly/3sq2vig
[10] Lorenzo Scalise, et al., “Wireless Sensing for the Respiratory Activity of Human Beings: Measurements and Wide-band Numerical Analysis,” Journal of Antennas and Propagation, vol. 2013, no. 396459, 2013.

What is claimed is:

1. An integrated methodology for performing at-home spirometry tests using improved analysis based on deep learning technology, comprising:
establishing a deep residual decoder network for patient lung function monitoring, and conducting curve learning with such decoder network based on a patient database of lung function measures;
providing a spirometry device outputting at least one key spirometry indicator and obtaining airflow data from a patient with incident airflow of a sample exhalation resulting in at least one key spirometry indicator from the spirometry device for the patient user; and
processing such spirometry indicator for the patient user using the decoder network to produce flow-volume graphing from such spirometry indicator to extend the capability of the spirometry device for finer-grained, long-term lung function monitoring.

2. Methodology as in claim 1, wherein:
the spirometry device outputs at least two key spirometry indicators;
the at least one key spirometry indicator for the patient user comprises at least two key spirometry indicators used as the input to the decoder network during said processing; and
said processing generates a complete flow-volume graph as the output from the decoder network.

3. Methodology as in claim 2, wherein the database comprises an open-source national database of lung function measures.

4. Methodology as in claim 3, wherein such open-source database comprises data from real but anonym ized patients collected over a period of time.

5. Methodology as in claim 4, wherein such open-source database comprises spirometry flow-volume and indicators measured from tests on diseased and healthy patients over different demographics.

6. Methodology as in claim 2, wherein:
the spirometry device outputs up to seven key spirometry indicators:
the input to the decoder network has up to seven key spirometry indicators from the spirometry device: and
the output from the decoder network comprises a flow-volume graph having resolution of at least down to 0.01 L.

7. Methodology as in claim 6, wherein the flow-volume graph has a maximum range of up to 10 L.

8. Methodology as in claim 3, wherein:
such database comprises data on over 100,000 patients; and
conducting curve learning includes feeding only spirometry indicator values from such database to the deep residual decoder network.

9. Methodology as in claim 1, wherein the deep residual decoder network includes use of deep stacked residual learning model architecture.

10. Methodology as in claim 9, wherein the decoder network includes a multi-stage architecture, with each stage of the decoder employing a fully-connected dense layer that expands the decoder input towards the full flow-volume graph output thereof.

11. Methodology as in claim 10, wherein the decoder network includes thirteen fully-connected layers stacked on each other, with each layer taking input from its previous layer to expand the compressed input towards the abstract features of the full flow-volume graph output, to expand the decoder's input to output.

12. Methodology as in claim 11, wherein the decoder network includes skip connections between the stacked layers that enable backpropagation and learning of residual mapping between input and output.

13. Methodology as in claim 12, wherein the decoder network uses ReLU (Rectified Linear Unit) activation function for its deep neural networks, with ReLU activation used for each stacked layer.

14. Methodology as in claim 13, wherein the decoder network uses a network loss function to appropriately tune the fully-connected layers' weights.

15. Methodology as in claim 14, wherein the decoder network architecture uses a Mean Absolute Error (MAE) as a loss function, which loss function calculates the average of the absolute difference between estimated values and real values, using Equation (1):
$$MAE = \frac{1}{N} {{\sum }^{N}}_{1} \vert {Y}_{i} - {\tilde{Y}}_{i} \vert \qquad ( 1 )$$
where, N is the total number of points, Yᵢ is the observed values and \tilde{Y}ᵢ is the predicted value.

16. Methodology as in claim 15, wherein the architecture uses an RMSprop optimizer, learning rate of 10⁻⁴, and mini-batch size of 32.

17. Methodology for implementing a deep stacked residual learning model to transform the capability of at-home spirometry testing devices using improved analysis based on deep learning technology to produce accurate flow-volume graphing from spirometry indicators, comprising:
establishing a decoder network for patient lung function monitoring, such network having deep stacked residual learning model architecture, and conducting curve learning with such decoder network based on spirometry indicators from a relatively large database of diverse patient lung function measures;
providing a spirometry device outputting a plurality of key spirometry indicators and obtaining airflow-based data from a patient with incident airflow of a sample exhalation resulting in at least one key spirometry indicator from the spirometry device for the patient user; and
processing such spirometry indicator data for the patient user using the decoder network to produce flow-volume graphing output from such spirometry indicator input to extend the capability of the spirometry device for finer-grained, long-term lung function monitoring.

18. Methodology as in claim 17, wherein the deep stacked residual learning model architecture includes a relatively large number of stacked layers to expand the decoder's input to output.

19. Methodology as in claim 18, wherein the decoder network includes thirteen fully-connected layers stacked on each other, with each layer taking input from its previous layer to expand the compressed input towards the abstract features of the full flow-volume graph output, to expand the decoder's input to output.

20. Methodology as in claim 17, wherein the decoder network includes a multi-stage architecture, with each stage of the decoder employing a fully-connected dense layer that expands the decoder input towards the full flow-volume graph output thereof.

21. Methodology as in claim 20, wherein the decoder network includes skip connections between the stacked layers that enable backpropagation and learning of residual mapping between input and output, to ensure accurate flow-volume graph prediction by the decoder network.

22. Methodology as in claim 21, wherein the decoder network uses ReLU activation function for its deep neural networks, with ReLU activation used for each stacked layer since the spirometry indicators do not have any negative values.

23. Methodology as in claim 22, wherein the decoder network uses a network loss function to appropriately tune the fully-connected layers' weights to ensure that data outliers in the database do not significantly adversely affect the network performance.

24. Methodology as in claim 23, wherein the decoder network architecture uses a Mean Absolute Error as a loss function, which loss function calculates the average of the absolute difference between estimated values and real values, using Equation (1):
$$MAE = \frac{1}{N} {{\sum }^{N}}_{1} \vert {Y}_{i} - {\tilde{Y}}_{i} \vert \qquad ( 1 )$$
where, N is the total number of points, Yᵢ is the observed values and \tilde{Y}ᵢ is the predicted value.

25. Methodology as in claim 24, wherein the architecture uses an RMSprop optimizer, learning rate of 10⁻⁴, and mini-batch size of 32.

26. Methodology as in claim 17, wherein:
the spirometry device outputs up to seven key spirometry indicators:
the input to the decoder network has up to seven key spirometry indicators from the spirometry device: and
the output from the decoder network comprises a flow-volume graph having resolution of at least down to 0.01 L.

27. Methodology as in claim 17, wherein the database comprises an open-source national database of lung function measures comprising data from real but anonym ized patients collected over a period of time from tests on both diseased and healthy patients over different demographics.

28. A smart device programmed with deep learning technology, for operating in accordance with claim 1 in conjunction with a spirometry device to produce flow-volume graphing from spirometry indicators from such spirometry device.

29. A device programmed with a deep stacked residual learning model operating in accordance with the methodology of claim 17, to transform the capability of at-home spirometry testing devices by using improved analysis to produce accurate flow-volume graphing based on spirometry indicators from an associated spirometry testing device.

30. A device for accessing deep learning technology, for operating in accordance with claim 1 in conjunction with a spirometry device to produce flow-volume graphing from spirometry indicators from such spirometry device.

31. A device as in claim 30, wherein the device is one of a smart device, a smartphone, and a device which accesses a cloud server.

32. A device having access to a deep stacked residual learning model operating in accordance with the methodology of claim 17, to transform the capability of at-home spirometry testing devices by using improved analysis to produce accurate flow-volume graphing based on spirometry indicators from an associated spirometry testing device.

33. A device as in claim 30, wherein the device is one of a smart device, a smartphone, and a device which accesses a cloud server.
